(19) United States
(12) Patent Application Publication
(10) Pub. No.: US 2022/0151995 A1
(43) Pub. Date: May 19, 2022

(54) PHARMACEUTICAL COMPOSITION FOR TREATING CANCER AND SUPPRESSING METASTASIS, CONTAINING SULFONAMIDE-BASED COMPOUND AS ACTIVE INGREDIENT

(71) Applicant: EXOSTEMTECH CO., LTD, Gyeonggi-Do (KR)

(72) Inventors: Moon-Chang BAEK, Suseong-gu (KR); Eun-Ju IM, Daegu (KR)

(21) Appl. No.: 17/562,424

(22) Filed: Dec. 27, 2021

Related U.S. Application Data
(63) Continuation-in-part of application No. 16/066,851, filed on Jun. 28, 2018.

(30) Foreign Application Priority Data
Dec. 31, 2015 (KR) 10-2015-0191448
Oct. 31, 2016 (KR) 10-2016-0143546

(51) Int. Cl. A61K 31/42; A61P 35/04

(52) U.S. Cl. CPC A61K 31/42; A61P 35/04

(57) ABSTRACT

The present invention relates to a method for treating cancer and/or suppressing metastasis in a subject, the method comprising administering an effective amount of a composition to a subject in need thereof, the composition comprising a sulfonamide-based compound as an active ingredient.

### CROSS-REFERENCE TO RELATED APPLICATIONS

This application is a continuation-in-part of U.S. patent application Ser. No. 16/066,851 filed Jun. 28, 2018, which is the U.S. National Phase Application under 35 U.S.C. § 371 of International Patent Application No. PCT/KR2016/014727, filed Dec. 15, 2016, and claims the priority of KR 10-2016-0143546, filed Oct. 31, 2016, and KR 10-2015-0191448, filed Dec. 31, 2015, which are incorporated by reference in their entireties. The International Application was published on Jul. 6, 2017, as International Publication No. WO 2017/116049 A1.

### TECHNICAL FIELD

The present invention relates to a pharmaceutical composition containing a sulfonamide-based compound as an active ingredient for treatment of cancer and inhibition of cancer metastasis and, more specifically, to novel uses of sulfonamide-based antibiotics sulfisoxazole or sulfadoxine having effects of preventing and treating cancer and inhibiting cancer metastasis.

### BACKGROUND ART

The present application claims priorities from Korean Patent Application No. 10-2015-0191448 filed with the Korean Intellectual Property Office on 31 Dec. 2015, and Korean Patent Application No. 10-2016-0143546 filed with the Korean Intellectual Property Office on 31 Oct. 2016, the disclosures of which are incorporated herein by reference.

Cancer collectively refers to a group of diseases, which mainly start with uncontrollable cell proliferation, leading to invasion and destruction of adjacent normal tissues or organs, and then creation of new growing sites therein, thereby finally taking the lives of individuals. Although a notable progress has been made over the past decade in controlling cell cycles or apoptosis and in seeking new targets including carcinogenic genes or cancer-suppressing genes in order to conquer cancer, the incidence of cancer has increased as the civilization has advanced.

As a method for treatment of cancer, chemotherapy, surgery, radiation therapy, and the like have been used. Of these, chemotherapy is a method using an anticancer drug and is the most commonly used for the treatment of cancer. Today, approximately 60 kinds of anticancer drugs are used in clinical practice, and novel anticancer drugs have been continuously developed along with an increase in knowledge about the occurrence of cancer and characteristics of cancer cells. However, most of the anticancer drugs currently used in clinical trials are chemically synthesized substances, which are often accompanied by side effects, such as nausea, vomiting, oral and intestinal ulcers, diarrhea, hair loss, and bone marrow suppression causing a deterioration in the production of blood active components. As examples of the side effects, it is known that Mitomycin C causes a renal failure and Adriamycin causes bone marrow suppression. Especially, cisplatin, which is one of the most useful drugs among the anticancer drugs that have been developed to date, is widely used for the treatment of testicular cancer, ovarian cancer, lung cancer, head and neck cancer, bladder cancer, gastric cancer, and cervical cancer, but has problems of the occurrence of side effects, for example, hematopoietic toxicity such as anemia, digestive toxicity such as vomiting and nausea, nephrotoxicity such as damage of kidney uriniferous tubules, hardness of hearing, electrolyte abnormality in the body, shock, abnormal peripheral nerves (R. T. Skeel, Handbook of Cancer Chemotherapy, pp. 89-91, 1999).

Therefore, a development of a novel anticancer drug having excellent safety and being capable of reducing side effects and toxicity involved in conventional anticancer drugs is urgently needed.

On the other hand, the concept of drug repositioning refers to an attempt to re-evaluate drugs that have already been developed, such as failed drugs in clinical trials or existing drugs on the market, and to discover novel drug efficacy of the drugs, thereby utilizing the drugs as therapeutics for other diseases. While the initial investment costs for the development of new drugs gradually increase, the proportion of failure greatly increases and the productivity and profitability sharply deteriorate. A drug repositioning strategy to overcome these crises may be a new paradigm in which the time and cost of drug development are reduced and the possibility of the success of development is increased by selecting drugs that have been already used in the art, whereby such a strategy is expected as an alternative for the development of new drugs. A good example is: Viagra, which was originally developed as a therapeutic for hypertension and angina but has been widely used as a therapeutic for erectile dysfunction; or aspirin, which has been shown to have efficacy on headaches but has recently been also shown to have efficacy on cardiovascular diseases.

Therefore, it may be preferable to discover and develop a new use of an existing drug, of which optimal doses and side effects have already been established, as an anticancer drug through drug repositioning since such drug repositioning can decrease the time and cost consumed in the development of drugs and can predict side effects in the development of anticancer drugs and thus can be directly applied in clinical trials.

### DETAILED DESCRIPTION OF THE INVENTION

### Technical Problem

The present inventors, while researching the possibility as anticancer drugs of 243 types of drugs associated with infectious diseases in the FDA-approved libraries, found that sulfonamide-based antibiotic sulfisoxazole or sulfadoxine has excellent effects of inhibiting proliferation, invasion, and migration of cancer cells, and thus completed the present invention.

Therefore, an aspect of the present invention is to provide a pharmaceutical composition for treating a cancer, the composition containing, as an active ingredient, a compound of Chemical Formula 1 or 2 below or a pharmaceutically acceptable salt thereof:

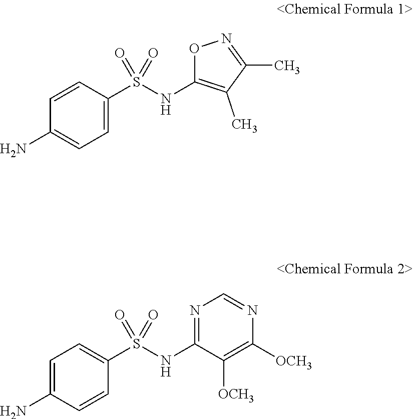 [SMILES: Cc1noc(NS(=O)(=O)c2ccc(N)cc2)c1C ; COc1ncnc(NS(=O)(=O)c2ccc(N)cc2)c1OC]

Another aspect of the present invention is to provide a pharmaceutical composition for inhibiting a cancer metastasis, the composition containing, as an active ingredient, a compound of Chemical Formula 1 or 2 below or a pharmaceutically acceptable salt thereof:

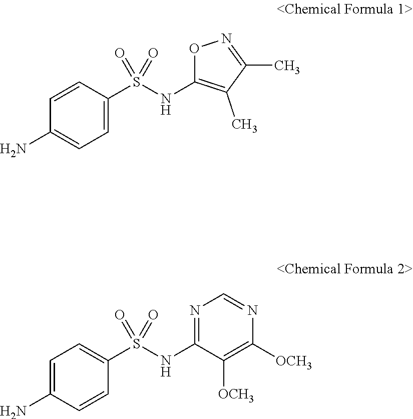 [SMILES: Cc1noc(NS(=O)(=O)c2ccc(N)cc2)c1C ; COc1ncnc(NS(=O)(=O)c2ccc(N)cc2)c1OC]

Another aspect of the present invention is to provide a use of a compound of Chemical Formula 1 or 2 below or a pharmaceutically acceptable salt thereof for preparing an agent for treating a cancer:

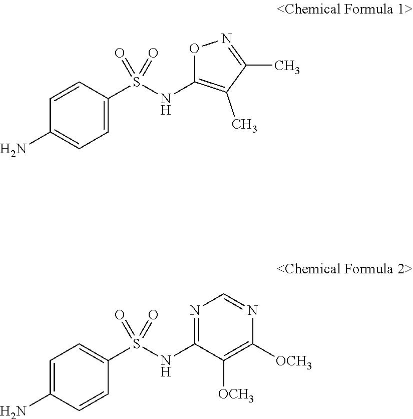 [SMILES: Cc1noc(NS(=O)(=O)c2ccc(N)cc2)c1C ; COc1ncnc(NS(=O)(=O)c2ccc(N)cc2)c1OC]

Another aspect of the present invention is to provide a method for treating a cancer in a subject in need thereof, the method comprising administering an effective amount of a composition containing, as an active ingredient, a compound of Chemical Formula 1 or 2 below or a pharmaceutically acceptable salt thereof a subject in need thereof:

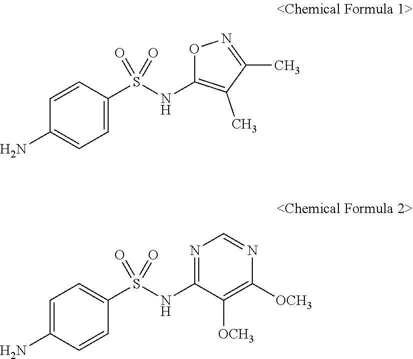 [SMILES: Cc1noc(NS(=O)(=O)c2ccc(N)cc2)c1C ; COc1ncnc(NS(=O)(=O)c2ccc(N)cc2)c1OC]

Another aspect of the present invention is to provide a use of a compound of Chemical Formula 1 or 2 below or a pharmaceutically acceptable salt thereof for preparing an agent for inhibiting a cancer metastasis:

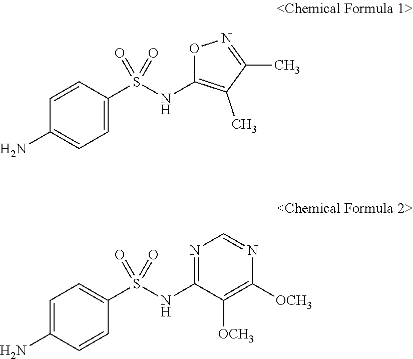 [SMILES: Cc1noc(NS(=O)(=O)c2ccc(N)cc2)c1C ; COc1ncnc(NS(=O)(=O)c2ccc(N)cc2)c1OC]

Another aspect of the present invention is to provide a method for inhibiting a cancer metastasis in a subject in need thereof, the method comprising administering an effective amount of a composition containing, as an active ingredient, a compound of Chemical Formula 1 or 2 below or a pharmaceutically acceptable salt thereof to a subject in need thereof:

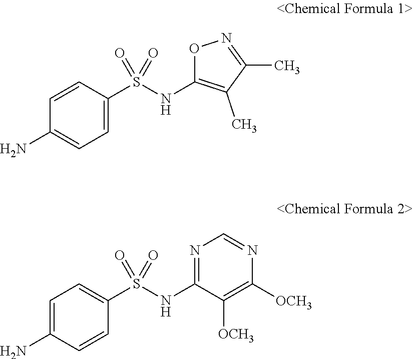 [SMILES: Cc1noc(NS(=O)(=O)c2ccc(N)cc2)c1C ; COc1ncnc(NS(=O)(=O)c2ccc(N)cc2)c1OC]

### Technical Solution

In accordance with an aspect of the present invention, there is provided a pharmaceutical composition for treating a cancer, the composition containing, as an active ingredient, a compound of Chemical Formula 1 or 2 below or a pharmaceutically acceptable salt thereof:

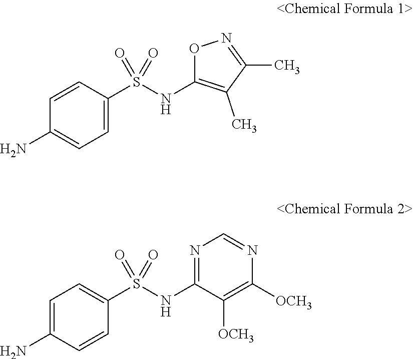 [SMILES: Cc1noc(NS(=O)(=O)c2ccc(N)cc2)c1C ; COc1ncnc(NS(=O)(=O)c2ccc(N)cc2)c1OC]

In accordance with another aspect of the present invention, there is provided a pharmaceutical composition for inhibiting a cancer metastasis, the composition containing, as an active ingredient, a compound of Chemical Formula 1 or 2 below or a pharmaceutically acceptable salt thereof:

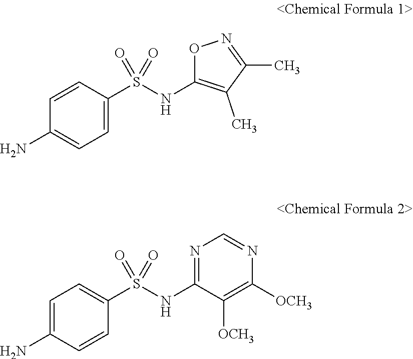 [SMILES: Cc1noc(NS(=O)(=O)c2ccc(N)cc2)c1C ; COc1ncnc(NS(=O)(=O)c2ccc(N)cc2)c1OC]

In accordance with another aspect of the present invention, there is provided a use of a compound of Chemical Formula 1 or 2 below or a pharmaceutically acceptable salt thereof for preparing an agent for treating a cancer:

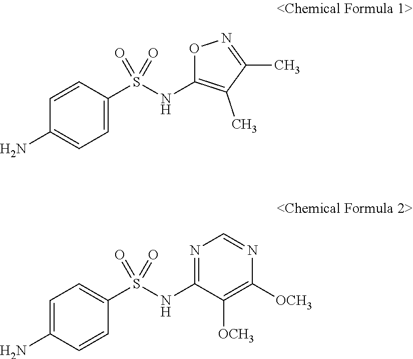 [SMILES: Cc1noc(NS(=O)(=O)c2ccc(N)cc2)c1C ; COc1ncnc(NS(=O)(=O)c2ccc(N)cc2)c1OC]

In accordance with another aspect of the present invention, there is provided a method for treating a cancer in a subject in need thereof, the method comprising administering an effective amount of a composition, the composition containing, as an active ingredient, a compound of Chemical Formula 1 or 2 below or a pharmaceutically acceptable salt thereof to a subject in need thereof:

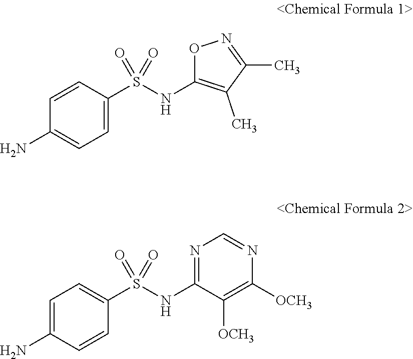 [SMILES: Cc1noc(NS(=O)(=O)c2ccc(N)cc2)c1C ; COc1ncnc(NS(=O)(=O)c2ccc(N)cc2)c1OC]

In accordance with another aspect of the present invention, there is provided a use of a compound of Chemical Formula 1 or 2 below or a pharmaceutically acceptable salt thereof for preparing an agent for inhibiting a cancer metastasis:

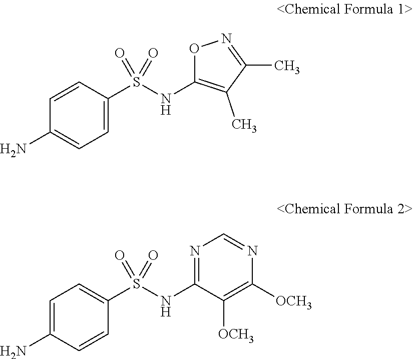 [SMILES: Cc1noc(NS(=O)(=O)c2ccc(N)cc2)c1C ; COc1ncnc(NS(=O)(=O)c2ccc(N)cc2)c1OC]

In accordance with another aspect of the present invention, there is provided a method for inhibiting a cancer metastasis in a subject in need thereof, the method comprising administering an effective amount of a composition, the composition containing, as an active ingredient, a compound of Chemical Formula 1 or 2 below or a pharmaceutically acceptable salt thereof to a subject in needed thereof:

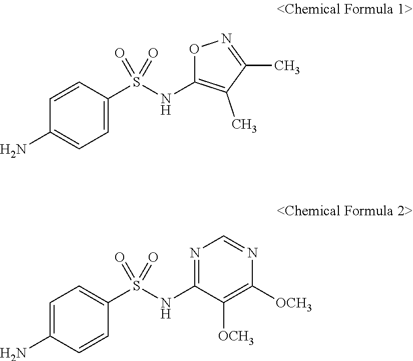 [SMILES: Cc1noc(NS(=O)(=O)c2ccc(N)cc2)c1C ; COc1ncnc(NS(=O)(=O)c2ccc(N)cc2)c1OC]

The compound of Chemical Formula 1 above is sulfisoxazole as a sulfonamide-based antibiotic, which is used as a drug showing broad antibacterial effects on gram-negative bacteria and gram-positive bacteria, and the compound can be prepared by a person skilled in the art according to a known preparation method. For example, the compound of Chemical Formula 1 can be prepared by a method disclosed in U.S. Pat. No. 2,430,094.

The compound of Chemical Formula 2 above is sulfadoxine as a sulfonamide-based antibiotic, which is a long-term persistent antibiotic that has been used mainly for treating malaria, but is currently not recommended due to a wide range of resistance thereof. The compound of Chemical Formula 2 can be prepared by a person skilled in the art according to a known method, and for example, may be prepared by a method known in U.S. Pat. No. 3,132,139.

Recently, anticancer effects of the sulfonamide-based compounds have been reported. Especially, it has been reported that (3-chloro-1H-indol-7-yl)-4-sulfamoylbenzenesulfonamide, N-(3-cyano-4-methyl-1H-indol-7-yl)-3-cyanobenzenesulfonamide, N-[[(4-chlorophenyl)amino]carbonyl]-2,3-dihydro-1H-indene-5-sulfonamide, N-[[(3,4-dichlorophenyl)amino]carbonyl]-2,3-dihydrobenzofuran-5-sulfonamide, N-(2,4-dichlorobenzoyl)-4-chlorophenylsulfonamide, N-(2,4-dichlorobenzoyl)-5-bromothiophene-2-sulfonamide, and 2-sulfanylamide-5-chloroquinoxilane show activity on several types of tumors, but antitumor activity of the compound represented by Chemical Formula 1 or 2 above has not been reported. Such antitumor activity is first revealed through the present invention.

According to an example of the present invention, it was confirmed that the compound of Chemical Formula 1 or 2 effectively inhibited the proliferation of MDA-MB231 cells as a breast cancer cell line and had very superior effects of inhibiting invasion and migration of cancer cells.

According to another example of the present invention, it was confirmed that the compound of Chemical Formula 1 effectively inhibited cancer growth to show a cancer treatment effect in in vivo animal models transplanted with cancer cells.

In the present invention, the compound of Chemical Formula 1 or 2 may be characterized by exerting effects of inhibiting proliferation, invasion, and migration of cancer cells by inhibiting the secretion of exosomes in cancer cells. Exosomes are secreted from almost all cells in the body, and especially, it has been known that the exosomes secreted in the cancer cells affect cancer differentiation, growth, metastasis, and angiogenesis in the progression of cancer. Therefore, the regulation of the secretion of these cancer cell-derived exosomes through drugs can inhibit cancer differentiation, growth, and metastasis.

According to an example of the present invention, it was confirmed that the compound of Chemical Formula 1 or 2 had effects of inhibiting secretion of exosomes in cells, and such effects are thought to result in the inhibition of proliferation, invasion, and migration of cancer cells.

As used herein, the term “cancer” refers to a disease associated with the control of apoptosis and means a disease occurring by hyper-proliferation of cells when the balance of normal apoptosis is broken. These abnormal hyperproliferative cells may sometimes invade into surrounding tissues and organs to form tumor masses and may cause the destruction or deformation of normal structures in the body, and such conditions are collectively referred to as cancer.

In general, a tumor refers to a mass abnormally grown by autonomous overgrowth of body tissues, and the tumors may be classified into benign tumors and malignant tumors. The malignant tumors have a much higher rate of growth than benign tumors, and the malignant tumors invade the surrounding tissues, resulting in metastasis, and threatening lives. Such malignant tumors are commonly referred to as “cancer”.

In the present invention, the kind of cancer is not particularly limited. Non-limited examples of the cancer may include cervical cancer, lung cancer, pancreatic cancer, non-small cell lung cancer, liver cancer, colon cancer, bone cancer, skin cancer, head cancer, neck cancer, skin melanoma, intraocular melanoma, uterine cancer, ovarian cancer, rectal cancer, liver cancer, brain tumor, bladder cancer, blood cancer, gastric cancer, perianal cancer, colon cancer, breast cancer, fallopian tube carcinoma, uterine endometrial carcinoma, vaginal cancer, vulva carcinoma, Hodgkin's disease, esophageal cancer, small intestine cancer, endocrine cancer, thyroid cancer, parathyroid cancer, adrenal gland cancer, soft tissue sarcoma, urethral cancer, penile cancer, prostate cancer, bladder cancer, kidney cancer, ureter cancer, renal cell carcinoma, renal pelvic carcinoma, central nervous system (CNS) tumor, primary CNS lymphoma, spinal tumor, brainstem glioma, or pituitary adenoma. Preferably, the cancer may be breast cancer, but is not limited thereto.

The pharmaceutical composition according to the present invention may contain the compound of Chemical Formula 1 or 2 of the present invention or a pharmaceutically acceptable salt thereof alone or may further contain at least one pharmaceutical acceptable carrier, vehicle, or diluent.

Examples of the pharmaceutically acceptable carrier may further include a carrier for oral administration or a carrier for parenteral administration. The carrier for oral administration may include lactose, starch, a cellulose derivative, magnesium stearate, stearic acid, and the like. Also, the carrier for parenteral administration may include water, suitable oil, a saline solution, aqueous glucose, and glycol, and may further include a stabilizer and a preservative.

Suitable examples of the stabilizer include an antioxidant, such as sodium hydrogen sulfite, sodium sulfite, or ascorbic acid. Suitable examples of the preservative include benzalkonium chloride, methyl- or propyl-paraben, and chlorobutanol. The following literature may be referred to for other examples of the pharmaceutically acceptable carrier (Remington's Pharmaceutical Sciences, 19th ed., Mack Publishing Company, Easton, Pa., 1995).

The pharmaceutical composition of the present invention may be administered to a mammal including a human being by any method. For example, the composition of the present invention may be administered orally or parenterally. The parental administration may include, but is not limited to, intravenous, intramuscular, intra-arterial, intramedullary, intradural, intracardiac, transdermal, subcutaneous, intraperitoneal, intranasal, intestinal, topical, sublingual, or rectal administration. Preferably, the pharmaceutical composition of the present invention may be orally administered.

The pharmaceutical composition of the present invention may be formulated as a preparation or an agent for oral administration or parental administration according to the route of administration as described above.

As for a preparation for oral administration, the composition of the present invention may be formulated in the forms of a powder, granules, a tablet, a pill, a sugar coated tablet, a capsule, a liquid, a gel, a syrup, a slurry, and a suspension, by the methods known in the art. For example, a tablet or sugar coated tablet for an oral preparation may be obtained by mixing an active ingredient with a solid excipient, pulverizing the mixture, adding a suitable adjuvant thereto, and then processing the mixture into a granule mixture. Suitable examples of the excipient may include: sugars including lactose, dextrose, sucrose, sorbitol, mannitol, xylitol, erythritol, and maltitol; starches including corn starch, wheat starch, rice starch, and potato starch; celluloses including cellulose, methyl cellulose, sodium carboxy methyl cellulose, and hydroxypropyl methyl cellulose; and fillers, such as gelatin and polyvinyl pyrrolidone. In some cases, cross-linked polyvinyl pyrrolidone, agar, alginic acid, or sodium alginate may be added as a disintegrant. Further, the pharmaceutical composition of the present invention may further contain an anti-coagulant, a lubricant, a wetting agent, a favoring agent, an emulsifier, and a preservative.

As for a preparation for parental administration, the composition of the present invention may be formulated in the forms of an injection, a cream, a lotion, an externally-applied ointment, an oil, a moisturizer, a gel, an aerosol, and a nasal inhaler, by the methods known in the art. These preparations are described in the literature, which is a formulary generally known in pharmaceutical chemistry (Remington's Pharmaceutical Science, 15th Edition, 1975. Mack Publishing Company, East on, Pa. 18042, Chapter 87: Blaug, Seyirour).

A total effective amount of the pharmaceutical composition of the present invention may be administered to a patient in a single dose, or may be administered in a multiple dose for a long period of time by a fractionated treatment protocol. The pharmaceutical composition of the present invention, the content of the active ingredient may vary depending on the severity of disease. A preferable dose of the present invention may be about 0.01 ug to 1,000 mg, and most preferably, 0.1 ug to 100 mg, per 1 kg of the body weight of a patient a day. However, as for a dosage of the pharmaceutical composition of the present invention, an effective dose to a patient is determined in consideration of various factors, such as route of administration, number of times of treatment, patient's age, body weight, health condition, and sex, severity of disease, food, and excretion rate, and thus considering these factors, a person skilled in the art could determine a proper effective amount of the composition of the present invention as a particular use as a cancer preventive and therapeutic agent. The pharmaceutical composition according to the present invention is not particularly limited to the dosage form, route of administration, and administration method thereof as long as the effects of the present invention are shown.

The pharmaceutical composition of the present invention may further contain a known anticancer drug or a known angiogenesis inhibitor, in addition to the compound of Chemical Formula 1 or 2 or the pharmaceutically acceptable salt thereof, and may be combined with other known therapies for the treatment of cancer. Other types of therapy include, but are not limited to, chemotherapy, radiation therapy, hormone therapy, bone marrow transplantation, stem-cell replacement therapy, other biological therapy, and immunotherapy.

Examples of anticancer drugs that may be contained in the pharmaceutical compositions of the invention include:

mechlorethamine, chlorambucil, phenylalanine, mustard, cyclophosphamide, ifosfamide, carmustine (BCNU), lomustine (CCNU), streptozotocin, busulfan, thiotepa, cisplatin, and carboplatin, as DNA alkylating agents; dactinomycin (actinomycin D), doxorubicin (adriamycin), daunorubicin, idarubicin, mitoxantrone, plicamycin, mitomycin, and C Bleomycin, as anticancer antibiotics; vincristine, vinblastine, paclitaxel, docetaxel, etoposide, teniposide, topotecan, and iridotecan, as plant alkaloids, but are not limited thereto.

In addition, the present invention provides a pharmaceutical composition for inhibiting a cancer metastasis, the composition containing, as an active ingredient, a compound represented by Chemical Formula 1 or 2 below or a pharmaceutically acceptable salt thereof:

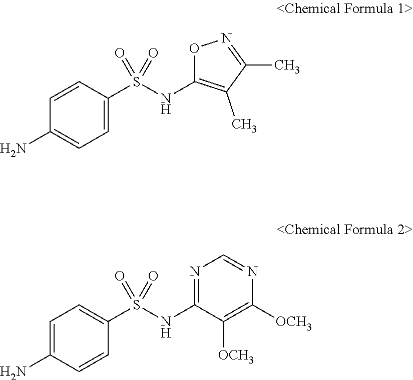 [SMILES: Cc1noc(NS(=O)(=O)c2ccc(N)cc2)c1C ; COc1ncnc(NS(=O)(=O)c2ccc(N)cc2)c1OC]

Cancer metastasis is the formation of new caner from the dissemination of cancer cells from primary cancer to other organs. Since metastasis is a major life-threatening condition in various cancer patients, the prevention or control of the metastasis is an important goal in cancer research. While surgery, chemotherapy, or radiotherapy is effective in the early cancer diagnosis without metastasis, the effectiveness of these therapies is reduced when there is metastasis at the time of diagnosis. In addition, metastasis is often confirmed during or after therapy even though metastasis was not confirmed at the time of diagnosis. The metastasis of cancer is very important in an aspect of clinical trials, but the procedure of metastasis is not yet fully understood.

Metastasis is composed of a series of stages: invasion, intravasation, arrest, extravasation, and colonization, and through this procedure, cancer cells disseminate from the primary organ, and finally, form cancer in other organs. The invasion as the first stage is an initiation stage of metastasis, and encompasses a change in the interaction between cancer cells or between a cancer cell and an extracellular matrix, disintegration of surrounding tissues, and migration of cancer cells into tissues, and the like. The intravasation as the second stage is that cancer cells pass through endothelial cells of blood vessels or lymphatic vessels and thus are included in the systemic circulation. Only a tiny portion of the imported cancer cells have been confirmed to survive in the circulation process. Some surviving cancer cells succeed in the extravasation, resulting in the permeation through capillary endothelial cells in other regions, and adapt to a new environment to proliferate, thereby forming metastatic cancer.

According to an embodiment of the present invention, the present inventors found through Matrigel invasion assay that the compound of Chemical Formula 1 or 2 significantly reduced the ability to invade the extracellular matrix of cancer cells. In order for cancer cells to metastasize to distant sites, cancer cells need to be able to invade from primary tumors to blood vessels and lymphatic vessels, and need to be able to again invade from the blood vessels and lymphatic vessels to the distant sites. These procedures are possible only if basement membranes surrounding the primary tumor and blood and lymphatic vessels are decomposed. Matrigel is composed of extracellular matrix components similar to components constituting basement membranes, so that the ability to invade the Matrigel can be utilized as a key indicator showing metastatic ability.

According to another example of the present invention, it was confirmed that the migration of cancer cells was remarkably reduced when the cancer cells were treated with the compound of Chemical Formula 1 or 2.

That is, the compound of Chemical Formula 1 or 2 has an excellent effect of inhibiting the metastasis of cancer cells by inhibiting the invasion and migration of cancer cells.

In the present invention, the pharmaceutical composition is as described above.

The present invention provides a use of a compound of Chemical Formula 1 or 2 below or a pharmaceutically acceptable salt thereof for preparing an agent for treating a cancer:

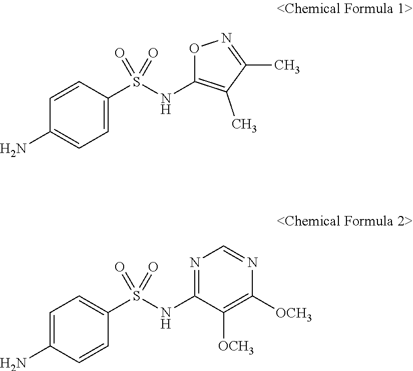 [SMILES: Cc1noc(NS(=O)(=O)c2ccc(N)cc2)c1C ; COc1ncnc(NS(=O)(=O)c2ccc(N)cc2)c1OC]

The present invention provides a method for treating a cancer in a subject in need thereof, the method comprising administering an effective amount of a composition containing, as an active ingredient, a compound of Chemical Formula 1 or 2 below or a pharmaceutically acceptable salt thereof to a subject in need thereof:

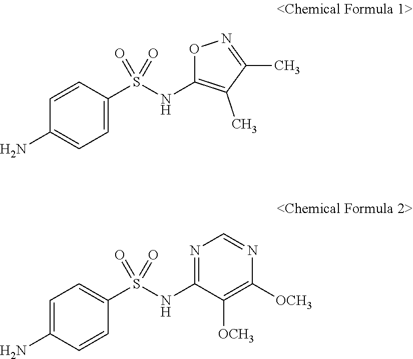 [SMILES: Cc1noc(NS(=O)(=O)c2ccc(N)cc2)c1C ; COc1ncnc(NS(=O)(=O)c2ccc(N)cc2)c1OC]

The present invention provides a use of a compound represented by Chemical Formula 1 or 2 below or a pharmaceutically acceptable salt thereof for preparing an agent for inhibiting a cancer metastasis:

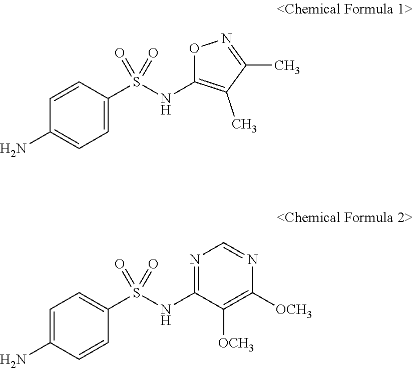 [SMILES: Cc1noc(NS(=O)(=O)c2ccc(N)cc2)c1C ; COc1ncnc(NS(=O)(=O)c2ccc(N)cc2)c1OC]

The present invention provides a method for inhibiting a cancer metastasis in a subject in need thereof, the method comprising administering an effective amount of a composition containing, as an active ingredient, a compound represented by Chemical Formula 1 or 2 below or a pharmaceutically acceptable salt thereof to a subject in needed thereof:

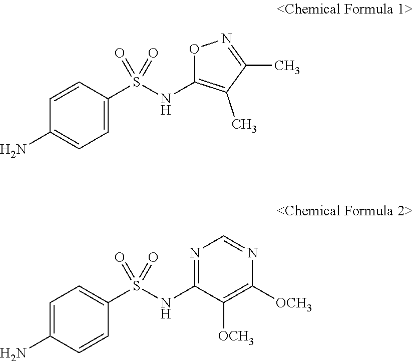 [SMILES: Cc1noc(NS(=O)(=O)c2ccc(N)cc2)c1C ; COc1ncnc(NS(=O)(=O)c2ccc(N)cc2)c1OC]

As used herein, the term “effective amount” refers to the amount exhibiting an effect of alleviating, treating, preventing, detecting, or diagnosing cancer or an effect of inhibiting or reducing the metastasis of cancer, and the term “subject” refers to an animal, preferably, a mammal, and especially, an animal including a human being, and may be a cell, tissue, and organ, or the like originating from an animal. The subject may be a patient in need of the effect.

As used herein, the term “treatment” broadly refers to alleviating cancer, a cancer-related disease, or a symptom of the cancer-related diseases, and may include healing, substantially preventing, or alleviating the condition of these diseases, and may include alleviating, curing, or preventing one or most of the symptoms resulting from cancer or cancer-related diseases, but is not limited thereto.

### Advantageous Effects

The sulfonamide-based compounds of the present invention are drugs which have excellent effects of inhibiting proliferation, invasion, and migration of cancer cells, in addition to an antibiotic effect known in the art, and of which the optimum doses or sides effects have already been established, and thus the compounds are highly likely to be utilized as compositions for prevention and treatment of cancer or inhibition of cancer metastasis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the results of evaluating the ability of sulfisoxazole to inhibit the proliferation of MDA-MB231 cancer cells.

FIG. 2 shows the results of evaluating the ability of sulfadoxine to inhibit the proliferation of MDA-MB231 cancer cells.

FIG. 3 shows the results of investigating the inhibitory effect of sulfisoxazole on cancer cell invasion through Matrigel invasion assay.

FIG. 4 shows the results of investigating the inhibitory effect of sulfadoxine on cancer cell invasion through Matrigel invasion assay.

FIGS. 5A and 5B show the results of evaluating the inhibitory effect of sulfisoxazole on cancer cell migration (FIG. 5A: microscopic observation results, FIG. 5B: T-scratch quantification results).

FIGS. 6A and 6B show the results of evaluating the inhibitory effect of sulfadoxine on cancer cell migration (FIG. 6A: microscopic observation results, FIG. 6B: T-scratch quantification results).

FIGS. 7A, 7B and 7C show the results of evaluating the inhibitory effect of sulfisoxazole (SFS) administration on cancer cell growth in MDA-MB231 cell-transplanted animal models (FIG. 7A: mean tumor size at the end of administration; FIG. 7B: tumor size of each mouse at the end of administration; FIG. 7C: average value of tumor sizes per mouse group over time).

FIG. 8 shows the results of evaluating the inhibitory effects of sulfisoxazole treatment on exosome secretion in melanoma cells SK-MEL-28, breast cancer cells MDA-MB 231, MCF7, and SK-BR3, colon cancer cells HCT116 and HT-29, lung cancer cells H157 and H460, prostate cancer cells LNCaP, and normal cells L132.

FIG. 9 shows the inhibition rate of exosome secretion of the selected ETA receptor antagonists.

FIG. 10 is a bar graph showing inhibition rate of exosome secretion of the selected ETA receptor antagonists by measuring the relative values of fluorescence.

FIG. 11 is a schematic diagram summarizing the method for evaluating the cancer exosome secretion.

FIG. 12 shows the exosome secretion from the cancer cells.

FIG. 13 is a schematic diagram summarizing the method for evaluating the cancer cell proliferation.

FIG. 14 shows the percentage of cancer cell proliferation.

FIG. 15 is a schematic diagram summarizing the in vivo experiment for evaluating the anticancer efficacy of SFX.

FIG. 16 shows the results of measuring tumor volume and weight.

FIG. 17 is a graph showing mouse weight and survival rate.

### MODE FOR CARRYING OUT THE INVENTION

Hereinafter, the present invention will be described in detail.

However, the following examples are merely for illustrating the present invention and are not intended to limit the scope of the present invention.

### Example 1

<1-1> Preparation of Samples

Sulfisoxazole was obtained from Sigma (31739), sulfadoxine was obtained from Sigma (S7821), and docetaxel was obtained from Sigma (01885). For cell tests, these substances were dissolved in dimethyl sulfoxide (DMSO) before use.

<1-2> Cell Culture

Melanoma cells SK-MEL-28, breast cancer cells MDA-MB 231, MCF7, and SK-BR3, colon cancer cells HCT116 and HT-29, lung cancer cells H157 and H460, prostate cancer cells LNCaP, and normal cells L132 were obtained from the American Tissue Culture Collection (ATCC). MDA-MB231, MCF7, HCT116, HT-29, and L132 cells were incubated in high-glucose DMEM (Hyclone) supplemented with 10% foetal calf serum (Hyclone), glutamine, penicillin, and streptomycin. SK-MEL-28 cells were incubated in MEM/EBSS (Hyclone) supplemented with 10% foetal calf serum (Hyclone), glutamine, penicillin, and streptomycin. SK-BR3, H157, H460, and LNCaP cells were incubated in RPMI (Hyclone) supplemented with 10% foetal calf serum (Hyclone), glutamine, penicillin, and streptomycin.

### Example 2

Inhibitory Effect on Cancer Cell Proliferation

MDA-MB231 cells were seeded in a 24-well plate at 1.times.10.sup.4 cells/well, and then had a time for cell stabilization through 12-hour incubation. After the 12-hour incubation, the cells were treated with sulfisoxazole and sulfadoxine at 25, 50, 100, and 200.mu.M, and then re-treated with the same drugs every hours. Thereafter, the cells were treated with MT tetrazolium reagent for each time, and then incubated for 24 hours. After 4 hours, the cell proliferation effect was investigated by measuring the absorbance at 495 nm of reduced MTT formazan (3-(4,5-dimethylthiazol-2-yl)-2,5-diphenyl-tetrazolium bromide). The docetaxel (10.mu.M), which is an anticancer drug currently in use, was used as a control.

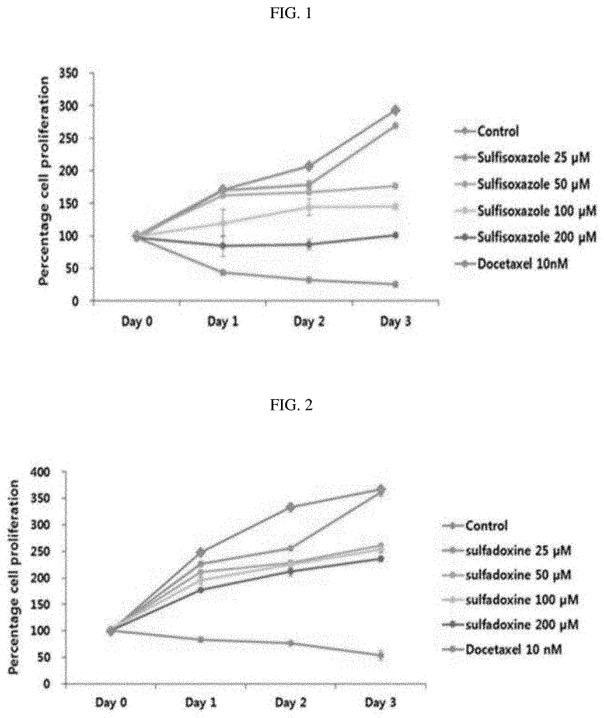

The results are shown in FIGS. 1 and 2.

As shown in FIG. 1, the proliferation of MDA-MB231 cell lines treated with sulfisoxazole was inhibited dependent on the concentration of sulfisoxazole. As shown in FIG. 2, the proliferation of MDA-MB231 cell lines treated with sulfadoxine was also significantly inhibited compared with the control.

### Example 3

Inhibitory Effect on Cancer Cell Invasion

A 8-pore size insert was placed in a 24-well plate, coated with serum-free medium and Matrigel, and then solidified at 37 for 4 hours. Then, 800.mu.1 of 10% FBS was added to the lower chamber and 1.times.10.sup.4 MDA-MB231 cells were added to the upper chamber, followed by treatment with sulfisoxazole or sulfadoxine at 25, 50, 100, and 200.mu.M. After 48 hours, the Matrigel in the insert was removed, followed by washing with PBS, and then the lower chamber was fixed with 60% ethanol, and then stained with crystal violet.

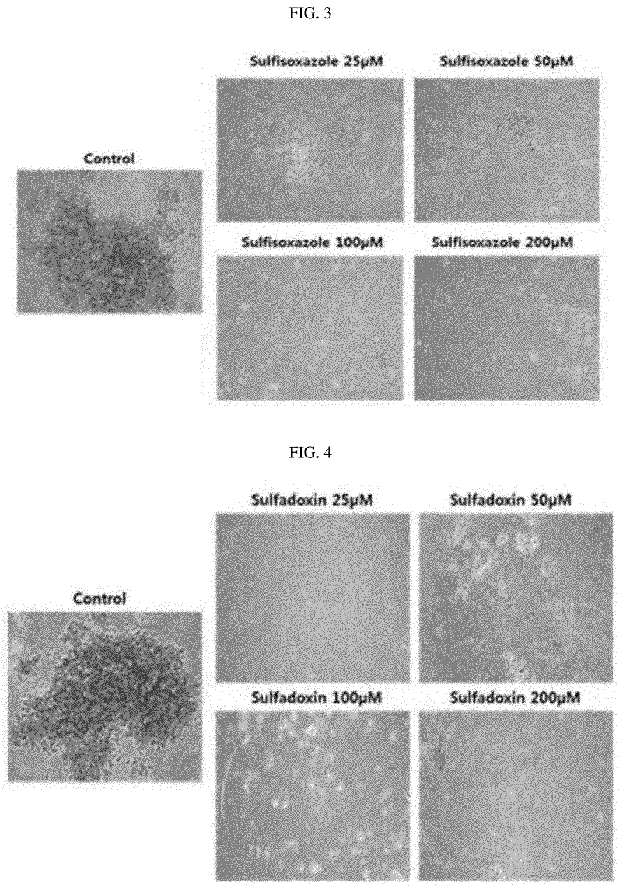

The results are shown in FIGS. 3 and 4.

As shown in FIGS. 3 and 4, it could be verified that the invasion of cancer cells was significantly inhibited in the groups treated with sulfisoxazole or sulfadoxine compared with the controls.

### Example 4

Inhibitory Effect of Cancer Cell Migration

MDA-MB231 cells were seeded in a 24-well plate at 2.times.10.sup.5 cells/well, and then incubated for 24 hours for the proliferation to a monolayer. After the incubation for 24 hours, the monolayer was straightly scratched with a yellow tip. The cells were washed with PBS to remove floating cells, and then photographed (0 h). Thereafter, a drug was added to a medium containing 2% serum, and then the cells were treated with the medium, and then incubated for 24 hours (24 h). After 24 hours, the cells were photographed for the same site to compare cell migration. The measurement of cell migration was carried out using T-scratch program.

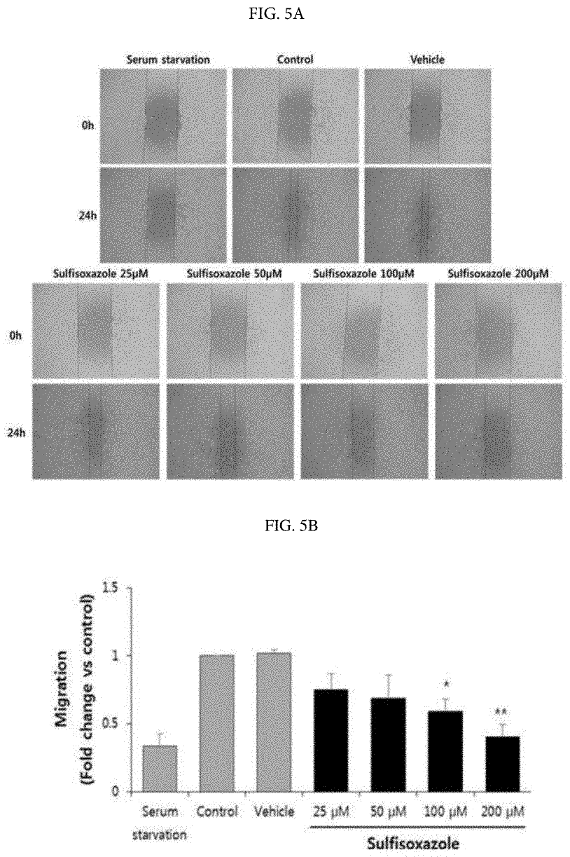

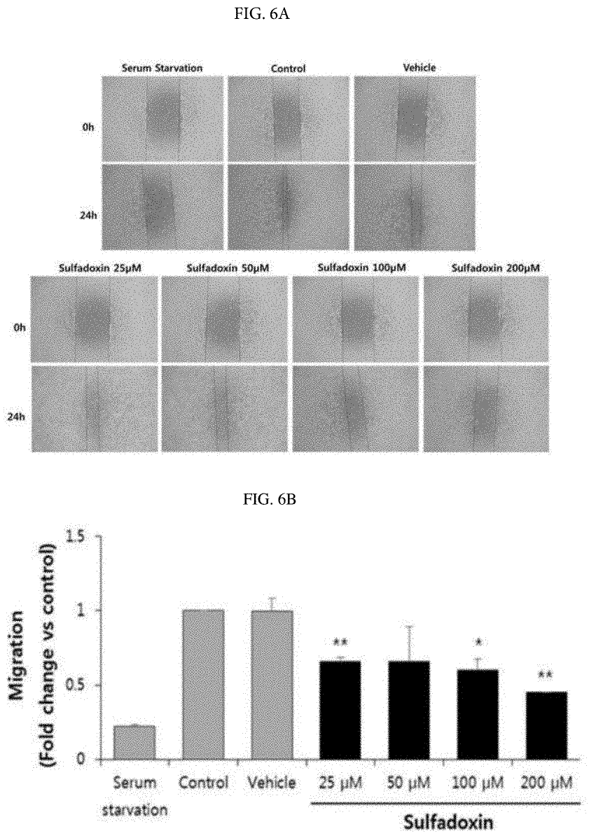

The results are shown in FIGS. 5 and 6.

As shown in FIGS. 5 and 6, it could be verified that many cancer cells in the control migrated to the center, but the migration of cancer cells in the groups treated with sulfisoxazole or sulfadoxine was inhibited dependent on the concentration of sulfisoxazole or sulfadoxine.

### Example 5

Anticancer Effect of Drug in Animal Models

The breast cancer cell lines, MDA-MB231 cells were seeded at 5.times.10.sup.6 cells into the subcutaneous tissues of Balb/c nude immunodeficient mice, and then the administration of a drug was started when the tumor mass reached 3-4 mm. Sulfisoxazole was administered orally in combination with saline 21 times total once a day, and the tumor volume was measured using a caliper once every three days. In addition, the tumor volume of each individual was measured on the last day of drug administration. The measurement values are shown in a graph.

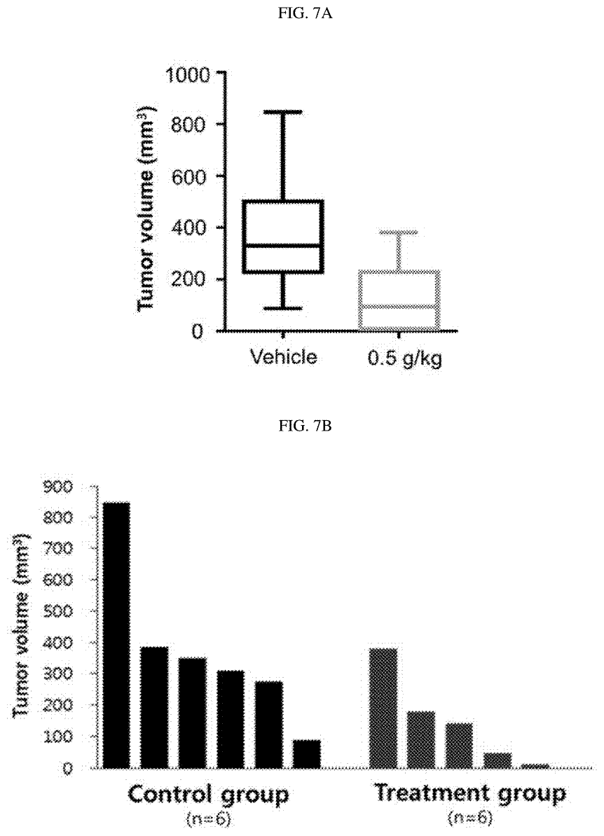

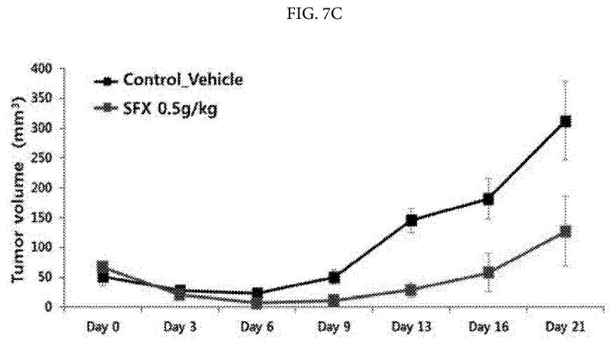

The results are shown in FIGS. 7A, 7B and 7C.

As shown in FIGS. 7A, 7B and 7C, it could be verified that the growth of cancer tissues was noticeably shown over time in mice administered with only saline, whereas the growth of cancer tissues was significantly reduced in mice administered with 0.5 g/kg of sulfisoxazole.

### Example 6

Inhibitory Effect of Exosome Secretion

Melanoma cells SK-MEL-28, breast cancer cells MDA-MB 231, MCF7, and SK-BR3, colon cancer cells HCT116 and HT-29, lung cancer cells H157 and H460, prostate cancer cells LNCaP, and normal cells L132 were seeded in a 15-cm plate at 5.times.10.sup.6, and then incubated for 24 hours. After the incubation for 24 hours, the medium was removed, and then the cells were washed with PBS, and treated with a drug at 25, 50, and 100 .mu.M in combination with serum-free medium. After 24-h incubation, the medium was take off, and then centrifuged at 300.times.g/3 min, 2,500.times.g/15 min, and 10,000.times.g/30 min. The supernatant was filtered using a 200-nm syringe filter, and then subjected to ultra-high speed centrifugation to give exosomes. Thereafter, the number of exosomes secreted by the cells was measured using the Nar-sight LM10 (Malvern) machine.

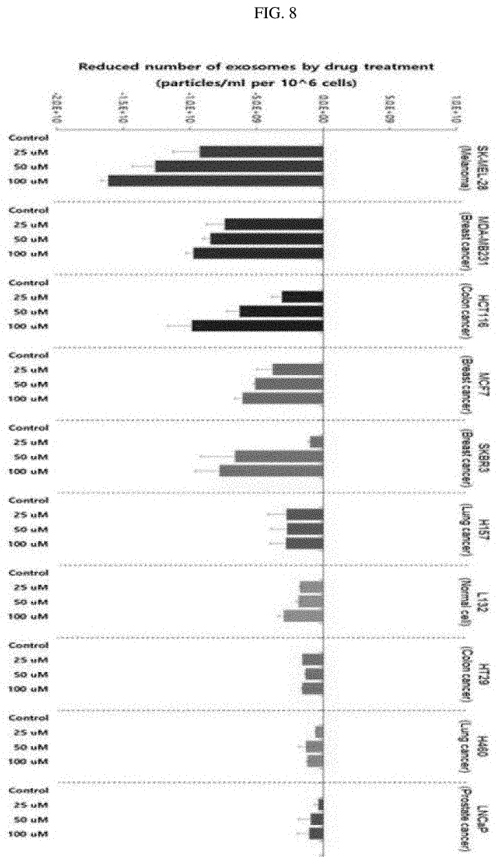

The results are shown in FIG. 8.

As shown in FIG. 8, it could be verified that exosome secretion was inhibited dependent on the concentration in the cells treated with the drug, compared with the control.

### Example 7

Inhibitory Effect of Exosome Secretion of Several ETA Receptor Antagonists

Among a total of 1,163 FDA-approved drug libraries, drugs effective in inhibiting exosome secretion of the MDA-MB231 triple negative breast cancer cell line were selected by carrying out screening twice. Sulfisoxazole, Sulfadiazine, Sulfathiazole and Ambrisentan, all of which are ETA receptor antagonists, were selected.

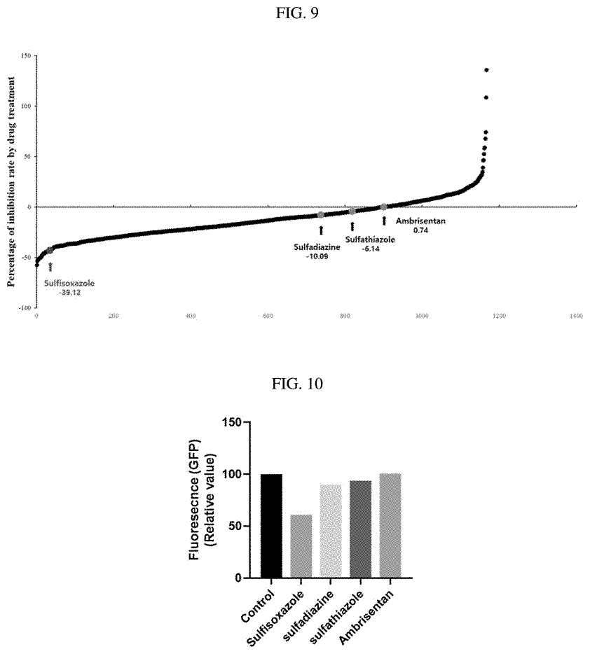

The percentage of inhibition rate of exosome secretion was evaluated for each of the selected ETA receptor antagonists. Briefly, MDA-MB231-CD63-GFP(+) cells were incubated with 30 pM of 1,163 individual FDA-approved drugs for 24 h. The fluorescence of each supernatant was measured at 485 nm (excitation) and 538 nm (emission) using a fluorescence microplate reader. Y-axis indicates the inhibition rate of exosome (CD63-GFP), and X-axis indicates the number of FDA-approved drugs. As shown in FIG. 9, Sulfisoxazole exhibited a significant improvement in the inhibition rate of exosome secretion relative to the other selected ETA receptor antagonists.

Among the 1,163 FDA-approved drugs, the exosome inhibition rate of ETA receptor antagonists is shown in FIG. 10. Sulfisoxazole showed remarkable exosome inhibition properties (60.8% of control group) than sulfadiazine (89.9%), sulfathiazole (93.8%), and ambrisentan (100.7%).

Thus, as shown in FIGS. 9 and 10, when MDA-MB231 cells were treated with the drugs, it was found that Sulfisoxazole was unexpectedly superior to the other ETA receptor antagonists in inhibiting exosome secretion.

### Example 8

Evaluation of Cancer Exosome Secretion Reduction

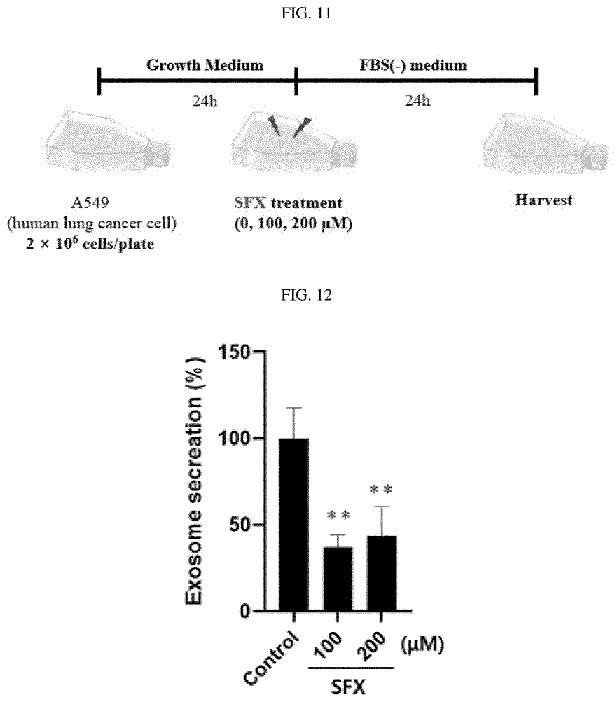

After seeding 2×10⁶ cells of A549 in T-flask, they were stabilized with growth medium for 24 hours. To isolate cancer exosomes, T-flask was treated with SFX at different concentrations (0, 100, 200 μM), and then lung cancer cells were cultured for one day. After the cell culture medium was collected and filtered, exosomes were separated through ultracentrifugation, and the number of exosomes was determined with Nanosight (FIG. 11). As shown in FIG. 12, it was found that the level of cancer exosome secretion in the group treated with SFX 100 μM and SFX 200 μM was significantly reduced compared to the control group (control, DMSO) (FIG. 12)

### Example 9

Evaluation of Cancer Cell Proliferation Reduction

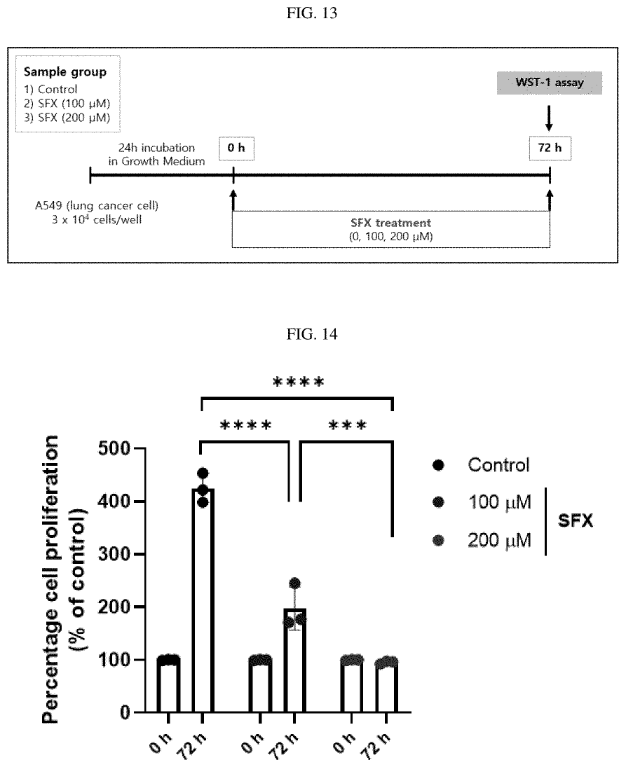

A549 cells were seeded in a 12-well plate at a concentration of 3×10⁴ cells/well and then stabilized with growth medium for 24 hours. To evaluate cancer cell growth inhibition, SFX was treated daily at different concentrations (0, 100, 200 μM). After 72 hours, cell growth rate was determined by WST-1 assay (FIG. 13). As shown in FIG. 14, it was found that the A549 cell growth was inhibited in a concentration-dependent manner in the group treated with SFX 100 μM and SFX 200 μM at 72 hours with high statistical significance, compared to the control group (control, DMSO).

### Example 10

In Vivo Test to Evaluate the Anticancer Efficacy of SFX

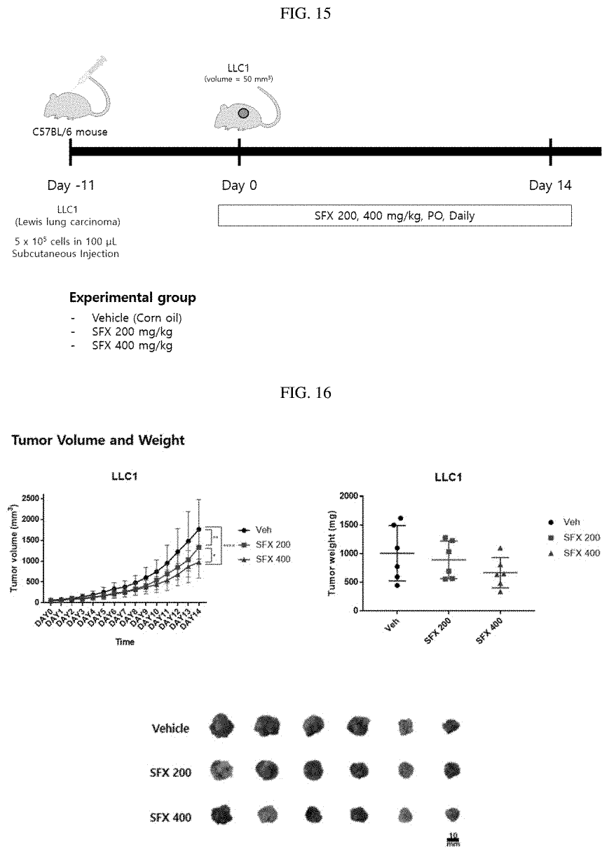

The cancer animal models were prepared on the last day acclimatization. C57BL/6 mice used for the production of LLC1 tumor mouse model were used as experimental animals, and after inoculation of LLC1 tumor cells in the mouse thigh at a concentration of 5×10⁵ cells/head, SFX was administered from the time when the tumor volume reached 50 mm³. Drug administration was carried out until the tumor volume of the vehicle administration group reached 1500 mm³ (FIG. 15).

After uniformly mixing the SFX solution prepared for each concentration by vortexing, a sonde for mice was connected to a 1 mL syringe to fill the solution, and 100 μL of the solution was orally administered to each mouse in each group.

Tumor volume was measured daily. The volume of the tumor was measured using a vernier caliper on the major axis and the minor axis, and the height was calculated as ½ the size of the minor axis.

[Tumor volume (mm3)=major axis (mm)×minor axis (mm)×½ of the minor axis]

In the groups administered with SFX 200 or 400 mg/kg, the tumor volume was significantly reduced at the end of the experiment compared to the control group (vehicle control), and the reduction in tumor volume was also significant according to the SFX concentration. The tumor weight was measured separately from the mouse at the end of the experiment, and the tumor weight showed a tendency to decrease in the SFX 200 and 400 mg/kg group compared to the control group (vehicle group). In addition, it was found that the tumor size was small in the SFX administration groups compared to the vehicle group (FIG. 16).

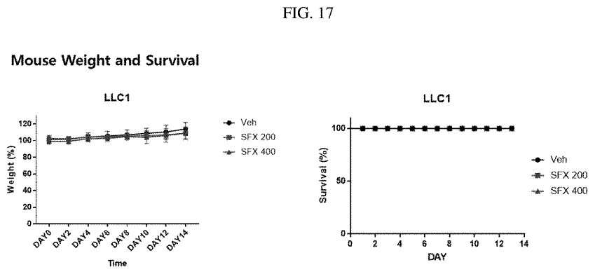

Mouse body weight was measured every other day from the start of the test, and was measured after oral administration of the drug. It was found that the body weight of the animals according to the SFX administration did not show a significant change. In addition, in the course of the experiment, there were no mice that died according to the SFX administration for each concentration (FIG. 17).

### INDUSTRIAL APPLICABILITY

The sulfonamide-based compounds of the present invention are drugs which have excellent effects of inhibiting proliferation, invasion, and migration of cancer cells, in addition to an antibiotic effect known in the art, and of which the optimum doses or sides effects have already been established, and thus the compounds are highly likely to be utilized as compositions for prevention and treatment of cancer or inhibition of cancer metastasis. Therefore, the sulfonamide-based compounds of the present invention are highly industrially applicable.

What is claimed is:

1. A method of inhibiting or treating a cancer, the method comprising administering an effective amount of a composition comprising, as an active ingredient, a compound defined by Chemical Formula 1 below or a pharmaceutically acceptable salt thereof to a subject in need thereof: 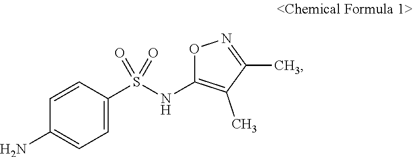 [SMILES: Cc1noc(NS(=O)(=O)c2ccc(N)cc2)c1C]
wherein the cancer is selected from the group consisting of melanoma, glioma, adrenal gland cancer, liver cancer and lung cancer.

2. The method of claim 1, wherein the cancer is melanoma.

3. The method of claim 1, wherein the compound defined by Chemical Formula 1 or a pharmaceutically acceptable salt thereof inhibits secretion of exosomes in the cancer.

4. The method of claim 1, wherein the compound defined by Chemical Formula 1 or a pharmaceutically acceptable salt thereof inhibits proliferation, invasion, and/or migration of the cancer.
